(19) United States
(12) Patent Application Publication
(10) Pub. No.: US 2019/0221309 A1
(43) Pub. Date: Jul. 18, 2019

(54) Coronary Artery Disease Screening Method by Using Cardiovascular Markers and Machine Learning Algorithms

(71) Applicants: Jang-Jih Lu, Taipei City (TW); Chun-Hsien Chen, Taoyuan City (TW); Hsin-Yao Wang, Chiayi City (TW); Yi-Hsin Chan, Taipei City (TW); Wei-Shang Shih, Taipei City (TW)

(72) Inventors: Jang-Jih Lu, Taipei City (TW); Chun-Hsien Chen, Taoyuan City (TW); Hsin-Yao Wang, Chiayi City (TW); Yi-Hsin Chan, Taipei City (TW); Wei-Shang Shih, Taipei City (TW)

(73) Assignees: CHANG GUNG MEMORIAL HOSPITAL, LINKOU, Taoyuan City (TW); Chang Gung University, Taoyuan City (TW); CATHAY GENERAL HOSPITAL, Taipei City (TW)

(21) Appl. No.: 15/871,159

(22) Filed: Jan. 15, 2018

(51) Int. Cl. G16H 50/20; G06F 19/24; G01N 33/50; G16H 50/30; G16H 50/50; C12Q 1/6883

(52) U.S. Cl. CPC G16H 50/20; G06F 19/24; G01N 33/5023; G01N 2800/50; G16H 50/50; C12Q 1/6883; G01N 2800/32; G16H 50/30

(57) ABSTRACT

A coronary artery disease (CAD) screening method includes 1) collecting clinical information of asymptomatic individuals and testing a plurality of samples of the individuals by using a cardiovascular markers panel including a plurality of cardiovascular markers; 2) entering the clinical information and the test results and the corresponding CAD states of the individuals into a machine learning platform; 3) selecting a plurality of roust variables from the clinical information and the cardiovascular markers of the cardiovascular markers panel by using feature selection methods; 4) using a machine learning algorithm embedded in the machine learning platform to establish a CAD prediction model; and 5) entering clinical information and sample data obtained by using the cardiovascular markers panel for an individual being screened into the CAD prediction model for calculation and analysis, thereby determining whether the individual being screened has CAD or not.

### BACKGROUND OF THE INVENTION

### 1. Field of the Invention

The invention relates to coronary artery disease (CAD) screening methods and more particularly to a coronary artery disease screening method by using cardiovascular markers and machine learning algorithms.

### 2. Description of Related Art

Deaths related to cardiovascular diseases are very high in many developing and developed countries. In particular, CAD may cause sudden cardiac death owing to acute coronary syndrome. Healing and caring for CAD patients can cause a great financial burden on the society. An early diagnosis of CAD can decrease the possibility of acute coronary syndrome, heart failure and other complications. However, simple CAD screening methods are not disclosed in the art. To the contrary, the conventional CAD screening technologies are disadvantageous owing to the factors of time consuming, high cost, radiation exposure, danger and manual determination.

For example, common CAD screening methods for asymptomatic people at risk of CAD include: cardiac nuclear medicine examination, cardiac catheterization and computed tomography coronary angiography. These methods aim to screen out CAD from people having no significant symptom. While these methods are effective, they have limitations. High radiation risk exists in cardiac nuclear medicine examination, cardiac catheterization and computed tomography coronary angiography. Cardiac catheterization has the highest accuracy but it has the risk of penetrating coronary arteries in operation. Computed tomography coronary angiography is a CAD screening method having a low invasiveness and a high accuracy. But it relies on computed tomography coronary angiography. Further, it has the problems of radiation exposure, high cost of equipment for computed tomography coronary angiography, high diagnosis cost and inappropriateness for large scale screening.

Another conventional CAD screening method involves a cardiovascular markers panel including many test values of the cardiovascular markers. Thus, a manual reading of the test values by a medical employee is required. The reading and interpretation of the test values are based on the threshold values of the cardiovascular markers. That is, a person being diagnosed may have a high risk of having CAD if the test value of any cardiovascular marker is greater than its corresponding threshold value. However, such method does not consider the comprehensive data distribution pattern of the cardiovascular markers as a whole. And in turn, it is not accurate and has a low performance in clinical use.

It is concluded that these conventional CAD screening methods are disadvantageous due to the drawbacks of inconvenience, high cost, and exposure to medical related damage and radiation.

Thus, the need for a practical, convenient and safe method for screening CAD of an ordinary people having no CAD symptom still exists.

### SUMMARY OF THE INVENTION

Therefore one object of the invention is to provide a coronary artery disease screening method for asymptomatic people at risk of CAD. The method comprises the following steps: 1). collecting clinical information of asymptomatic individuals including sex, age, Body Mass Index (BMI), hypertension status, as well as diabetes mellitus status, and testing a plurality of samples of the individuals by using a cardiovascular markers panel including a plurality of cardiovascular markers; 2). entering clinical information and test results of the individuals and their corresponding CAD states; 3). selecting a plurality of robust variables from the clinical information and the cardiovascular markers by using feature selection methods; 4). using a machine learning algorithm to establish a CAD prediction model; and 5). entering the clinical information and sample data obtained by using the cardiovascular markers panel for an individual being screened into the CAD prediction model for calculation and analysis, thereby determining whether the individual being screened has CAD or not.

The above and other objects, features and advantages of the invention will become apparent from the following detailed description taken with the accompanying drawings.

### BRIEF DESCRIPTION OF THE DRAWINGS

FIG. 1 is a flowchart of the CAD screening method according to the invention; and

FIG. 2 is a chart showing CAD prediction performance by using single cardiovascular marker or a cardiovascular markers panel combined with machine learning algorithms in terms of the area under the receiver operating characteristic (ROC) curve.

### DETAILED DESCRIPTION OF THE INVENTION

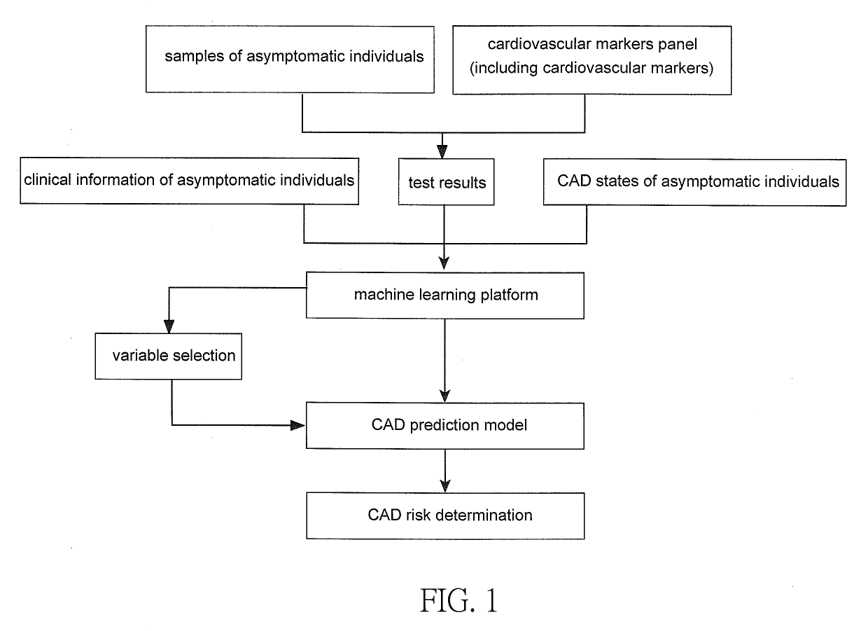

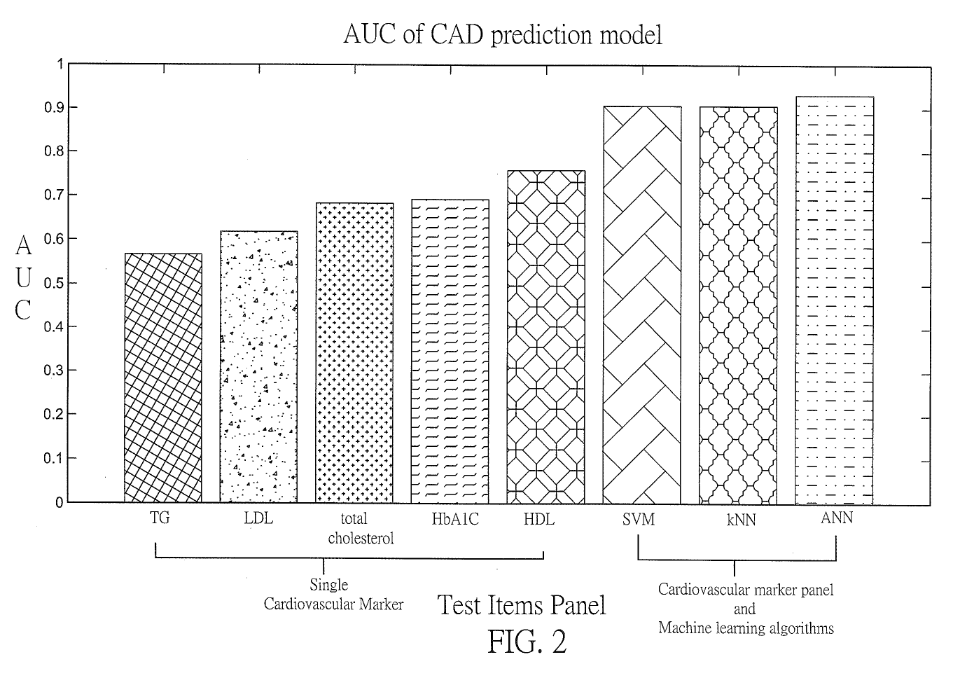

Referring to FIGS. 1 and 2, a CAD prediction model established in accordance with the invention comprises the following steps as described in detail below.

First, clinical information of asymptomatic individuals including sex, age, Body Mass Index (BMI), hypertension status, as well as diabetes mellitus status are collected, and samples such as blood, urine, saliva, sweat, feces, pleural fluid, and ascites fluid or cerebrospinal fluid of the individuals are tested by using a cardiovascular markers panel. Clinical information, test results and the corresponding CAD states of the individuals are entered into a machine learning platform. The CAD state is classified based on having CAD or not. Alternatively, the CAD state is classified based on the degree of severity of CAD. Next, a variable selection method is used in the machine learning platform to select robust variables from the clinical information and the cardiovascular markers of the panel. Next, a machine learning algorithm is used to establish a CAD prediction model. Finally, clinical information and sample data obtained by using the cardiovascular markers panel for an individual being screened is entered into the CAD prediction model for calculation and analysis. As a result, it is possible of determining whether the individual being screened has CAD or not. If the determination by the CAD prediction model is positive (i.e., the individual being screened having a high probability of having CAD), the individual being screened will be notified so that the individual being screened may take further actions including other examinations for CAD confirmation and consultation with a physician about CAD treatment.

It is noted that the length of time between the date of determining the CAD state and the date of taking the test by using the cardiovascular markers is from one day to three years depending on different applications.

The cardiovascular markers panel includes High Density Lipoprotein (HDL), Low Density Lipoprotein (LDL), Triglycerol (TG), total cholesterol, blood sugar, micro-albumin, glycosylated hemoglobin (HbA1C), High-Sensitivity C-Reactive Protein (hsCRP), Homocysteine, lipoprotein, uric acid, cardiac troponins, creatine kinase (CK), N-terminal Pro Brain Natriuretic Peptide (NT ProBNP), B-type Natraretic Peptide (BNP), N-terminal Pro Brain Natriuretic Peptide (NT ProBNP), procalcitonin (PCT), erythrocyte sedimentation rate (ESR), lactic dehydrogenase (LDH), Na⁺, K⁺, Ca²⁺, Cl⁻, Mg²⁺, Fe2+, Fe³⁺, Urea Nitrogen, Creatinine, Cystatin C, Bilirubin, Ketone and pH.

An embodiment is detailed below.

Conditions (including admission and exclusion) of an individual being screened and the number of samples:

An adult of at least 20-year old is appropriate for taking the test of the cardiovascular markers panel. Medical records of patients are checked to find 543 potential candidates. Thus, there is no need of recruiting candidates.

Design and Method:

Clinical information, test items and measurements include sex, age, Body Mass Index (BMI), Hypertension status, Diabetes mellitus status, High Density Lipoprotein (HDL), Low Density Lipoprotein (LDL), Triglycerol (TG), and glycosylated hemoglobin (HbA1C). There are 543 candidates and blood drawing and cardiac catheterization are conducted on each candidate in order to determine their CAD state.

Feature selection: after preliminary data cleaning, an univariate statistics is conducted in the embodiment. An appropriate univariate statistics (e.g., Chi-square test or t test) is selected based on the characteristic of the variables. As a result, variables including sex, BMI, diabetes mellitus status, hypertension status, TG, low density lipoprotein, total cholesterol, HbA1C and high density lipoprotein are selected as features of subsequent model training.

However, univariate statistics belong to filter methods for variable selection. Wrapper methods, embedded methods, and other filter methods can also be applied to the selection of robust variables from the clinical information and optimum cardiovascular markers of the cardiovascular markers panel.

After the feature selection, a plurality of CAD prediction models are established by machine learning algorithms in the embodiment, and the machine learning algorithms include k-nearest neighbors, k Nearest Neighbor (kNN), Support Vector Machines (SVM) and Artificial Neuron Network (ANN).

Retrospective period of the embodiment: from Sep. 1, 2010 to Mar. 31, 2011.

Result Evaluation and Statistical Method:

In the embodiment, data distributions of the cardiovascular markers are calculated. Further, prediction models are trained based on the selected variables and their values. In the embodiment, 5-fold cross-validation is used to evaluate the prediction performance of each prediction model. Performance of the prediction model is evaluated based on the ROC curve and the area under the curve (AUC) is calculated accordingly.

FIG. 2 is a chart showing the CAD prediction performance of various prediction models in terms of AUC. The AUCs of CAD prediction models established by single cardiovascular markers (namely, TG, low density lipoprotein, total cholesterol, HbA1C or high density lipoprotein) and the AUCs of CAD prediction models established by the cardiovascular markers panel combined with different machine learning algorithms (namely, SVM, kNN or Artificial Neural Network) are used to evaluate the CAD prediction performance. From the figure, it is shown that the AUC of the prediction model using a single cardiovascular marker is about 0.7 at most. However, for a prediction model using one of the machine learning algorithms to analyze the cardiovascular markers panel (including a plurality of cardiovascular markers), the CAD prediction AUC can be greatly increased to about 0.9. Thus, using machine learning algorithms to integrate and learn the data of the cardiovascular markers panel can greatly increase the performance of CAD screening.

It is concluded that the invention has the following characteristics and advantages: The cardiovascular markers panel can obtain test results of a plurality of cardiovascular markers in a single blood test for asymptomatic individuals being screened for CAD. Integrating clinical information and the test data of the cardiovascular markers with machine learning algorithms allows comprehensive analysis of the distribution difference between CAD and non-CAD cases. The trained CAD prediction model can be easily copied to users' computers for use. Thus, it can be widely used in CAD screening. Therefore, it contributes greatly to the advancement of medical diagnosis. Further, its accuracy, time efficiency, cost effectiveness and repeatability in comparison with the conventional manual reading methods are greatly improved. Further, invasiveness and risk of radiation exposure are greatly decreased compared to the conventional CAD screening methods.

While the invention has been described in terms of preferred embodiments, those skilled in the art will recognize that the invention can be practiced with modifications within the spirit and scope of the appended claims.

What is claimed is:

1. A coronary artery disease screening method comprising the steps of:
(a) collecting clinical information of asymptomatic individuals, and testing a plurality of samples of the individuals by using a cardiovascular markers panel including a plurality of cardiovascular markers;
(b) entering the clinical information, the test results and corresponding CAD states of the individuals into a machine learning platform;
(c) selecting a plurality of robust variables from the clinical information and cardiovascular markers of the cardiovascular markers panel by using feature selection methods;
(d) using a machine learning algorithm to establish a CAD prediction model; and
(e) entering clinical information and the sample data obtained by using the cardiovascular markers panel for an individual being screened into the CAD prediction model for calculation and analysis, thereby determining whether the individual being screened has CAD or not.

2. The method of claim 1, wherein in step (e) if it is determined that the individual being screened having a high probability of having CAD, the individual being screened will be notified.

3. The method of claim 1, wherein in step (b) the CAD state is classified based on either having CAD or not, or degree of severity of CAD.

4. The method of claim 1, wherein the length of time between the date of determining the CAD state and the date of taking the test by using the cardiovascular markers is from one day to three years.

5. The method of claim 1, wherein the cardiovascular markers panel includes High Density Lipoprotein (HDL), Low Density Lipoprotein (LDL), Triglycerol (TG), total cholesterol, blood sugar, microalbumin, glycosylated hemoglobin (HbA1C), High-Sensitivity C-Reactive Protein (hsCRP), Homocysteine, lipoprotein, uric acid, cardiac troponins, creatine kinase (CK), N-terminal Pro Brain Natriuretic Peptide (NT ProBNP), B-type Natraretic Peptide (BNP), N-terminal Pro Brain Natriuretic Peptide (NT ProBNP), procalcitonin (PCT), erythrocyte sedimentation rate (ESR), lactic dehydrogenase (LDH), Na⁺, K⁺, Ca²⁺, Cl⁻, Mg²⁺, Fe²⁺, Fe³⁺, Urea Nitrogen, Creatinine, Cystatin C, Bilirubin, Ketone and pH.

6. The method of claim 1, wherein in step (c) the selection of the robust variables from the clinical information and optimum cardiovascular markers of the cardiovascular markers panel is done by univariate statistics embedded in the machine learning platform. However, univariate statistics belong to filter methods for variable selection. Wrapper methods, embedded methods, and other filter methods can also be applied to the selection of robust variables from the clinical information and optimum cardiovascular markers of the cardiovascular markers panel.

7. The method of claim 6, wherein the univariate statistics are Chi-square test and t-test.

8. The method of claim 1, wherein in step (c) the optimum selected cardiovascular marker variables are sex, age, Body Mass Index (BMI), hypertension status, diabetes mellitus status, TG, High Density Lipoprotein (HDL), Low Density Lipoprotein (LDL), total cholesterol, and glycosylated hemoglobin (HbA1C).

9. The method of claim 1, wherein in step (a) the clinical information is including sex, age, Body Mass Index (BMI), hypertension status, and diabetes mellitus status.

10. The method of claim 1, wherein in step (a) the samples are the body fluids includes blood, urine, saliva, sweat, feces, pleural fluid, and ascites fluid or cerebrospinal fluid.

11. The method of claim 1, wherein each of the machine learning algorithms is a Logistic Regression, a k-Nearest Neighbor, a Support Vector Machine, an Artificial Neural Network, a Decision Tree, a Random Forest, a Bayesian Network, or any combinations thereof.
